(19) United States
(12) Patent Application Publication
(10) Pub. No.: US 2023/0098763 A1
(43) Pub. Date: Mar. 30, 2023

(54) COUPLING MEMBER AND MEASUREMENT SYSTEM

(71) Applicant: NejiLaw Inc., Bunkyo-ku, Tokyo (JP)

(72) Inventor: Hiroshi Michiwaki, Bunkyo-ku, Tokyo (JP)

(21) Appl. No.: 17/785,389

(22) PCT Filed: Dec. 14, 2020
(86) PCT No.: PCT/JP2020/046484

§ 371 Date: Jun. 14, 2022

(30) Foreign Application Priority Data
Dec. 17, 2019 (JP) 2019-227493

(51) Int. Cl. G01B 21/32; G01L 5/00; G01M 5/00

(52) U.S. Cl. CPC G01B 21/32; G01L 5/0047; G01M 5/0041

(57) ABSTRACT

A coupling member for coupling a solidified body and a structural member includes a sensor part that is capable of measuring physical variation resulting from external force and is for detecting information that will help determine whether there is an abnormality in the solidified body and/or structural member. The coupling member has an embedded part at one end that is embedded in the solidified body and/or ground and has a fixing part that is on the side of the other end extending outside of the solidified body and is capable of fixing the structural member.

### CROSS-REFERENCE TO RELATED APPLICATIONS

This application is a national phase entry under 35 U.S.C. § 371 of International Patent Application PCT/JP2020/046484, filed Dec. 14, 2020, designating the United States of America and published as International Patent Publication WO 2021/125126 A1 on Jun. 24, 2021, which claims the benefit under Article 8 of the Patent Cooperation Treaty to Japanese Patent Application Ser. No. 2019-227493, filed Dec. 17, 2019.

### TECHNICAL FIELD

The following description relates to a method of measuring information on each part of a structure such as a building or a bridge, particularly information on stress applied on peripheral parts of a foundation.

### BACKGROUND

Currently, there are various structures related to social infrastructure, such as school buildings, stations buildings, airport terminals, hospitals, government buildings for municipalities, bridges, and tunnels. Although such the structures are to be used for a long period of time, deterioration is inevitable due to exposure to an external force by impacts, such as deterioration over time and an earthquake. If the deterioration is neglected, there is also a risk of man-made disasters.

Therefore, it is becoming important to strengthen social infrastructure including the structures through maintenance and also to reduce and prevent disasters (National Resilience) for the future.

### BRIEF SUMMARY

Nowadays, however, a large number of structures exist, making it practically difficult to give priority to the structures to be maintained or to determine which part of a structure should be intensively maintained.

But even now, records (Karte) for bridge management are still being written, and the bridges are regularly inspected by a person in charge in municipalities and Prefectures in Japan, in order to maintain and manage the bridge. However, the inspection is conducted mainly based on visual inspection by a human being, thereby causing difference among individuals while lacking objectivity. Thus, there has been an issue that it is hard to use the inspection in determining the fundamental maintenance.

The present disclosure has been made by earnest research in consideration of the above issues. The present disclosure enables objective measurement of the condition of a structure, intended to be applied for the judgment of the maintenance time and the design of a better structure.

According to an aspect, there is provided a coupling member for coupling a solidified body and a structural member, having a sensor part configured to be capable of measuring a physical change due to an external force and detect information that helps determine abnormalities in the solidified body and/or the structural member. The coupling member includes an embedded part at one end embedded in the solidified body and/or a ground, and a fixing part configured to be capable of fixing the structural member to the other end side extending outside the solidified body.

Further, in the coupling member of an example embodiment of the present disclosure, the sensor part is installed in a surface layer region of a boundary peripheral portion of the solidified body and the outside of the solidified body.

Furthermore, in the coupling member of an example embodiment of the present disclosure, the sensor part is installed a region inside the solidified body of a boundary peripheral portion of the solidified body and the outside of the solidified body.

According to another aspect, there is provided a measurement system including the coupling member and an information collection device that is connected to the sensor part by wire or wirelessly and configured to accumulate measurement information measured by the sensor part and determine abnormality in the solidified body and/or the structural member based on the measurement information.

According to the present disclosure, it is possible to objectively monitor stress, warping, displacement, and the like, occurring in a structure.

### BRIEF DESCRIPTION OF THE DRAWINGS

While the specification concludes with claims particularly pointing out and distinctly claiming embodiments of the present disclosure, the advantages of embodiments of the disclosure may be more readily ascertained from the following description of embodiments of the disclosure when read in conjunction with the accompanying drawings in which:

FIG. 1 is a diagram illustrating a whole configuration of a measurement system according to an example embodiment.

FIG. 2 is a perspective view illustrating an enlarged structural body of a structure to which the measurement system is applied according to an example embodiment.

FIG. 3 is a diagram illustrating an anchor bolt as a screw member according to an example embodiment.

FIG. 4 is a cross-sectional view illustrating a configuration of a cylindrical part according to an example embodiment.

FIG. 5 is a perspective view illustrating an example of disposition of a sensor pattern, a conductive path, and a terminal of an anchor bolt according to an example embodiment.

FIG. 6 is a block diagram illustrating a configuration of a circuit board according to an example embodiment.

FIG. 7 shows (A) a block diagram illustrating a hardware configuration of an information collection device of the measurement system and (B) a functional configuration of the information collection device according to an example embodiment.

FIG. 8 is a sectional diagram illustrating a first female screw body and a second female screw body screwed to an anchor bolt according to an example embodiment.

FIG. 9 is diagrams illustrating examples of a disposition site of a sensor pattern according to an example embodiment.

FIG. 10 is diagrams illustrating an anchor bolt having two sensor patterns according to an example embodiment.

### DETAILED DESCRIPTION

Hereinafter, an example embodiment of the present disclosure will be described in detail with reference to the drawings.

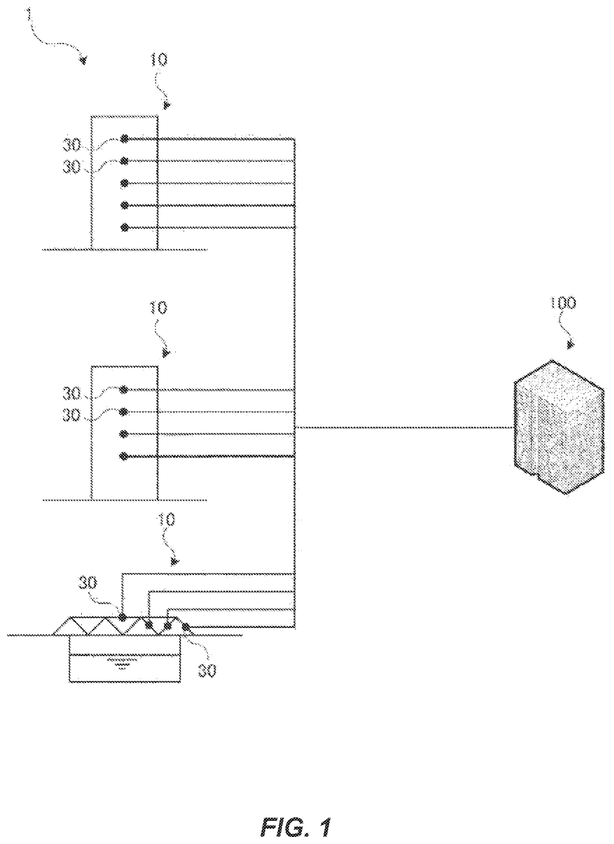

FIG. 1 illustrates a measurement system 1 of a structure according to an example embodiment of the present disclosure. The measurement system 1 is configured to include a plurality of structures 10 such as a building or a bridge, an anchor member (coupling member) 30 configured to be used as a member during construction of the structure 10, and an information collection device 100 configured to be connected by wire or wirelessly with respect to the anchor member 30.

The anchor member 30 may be a male screw body, a female screw body, or an anchor having one portion formed with a rod-like body such as a screw part or reinforcing bar, preferably used in a basic structural member of the structure 10, secondary concrete products such as mortar, concrete foundation, or precast, and structural members erected on solidified bodies (crust-shaped sintered bodies) such as glass and resins, and is directly embedded in the ground.

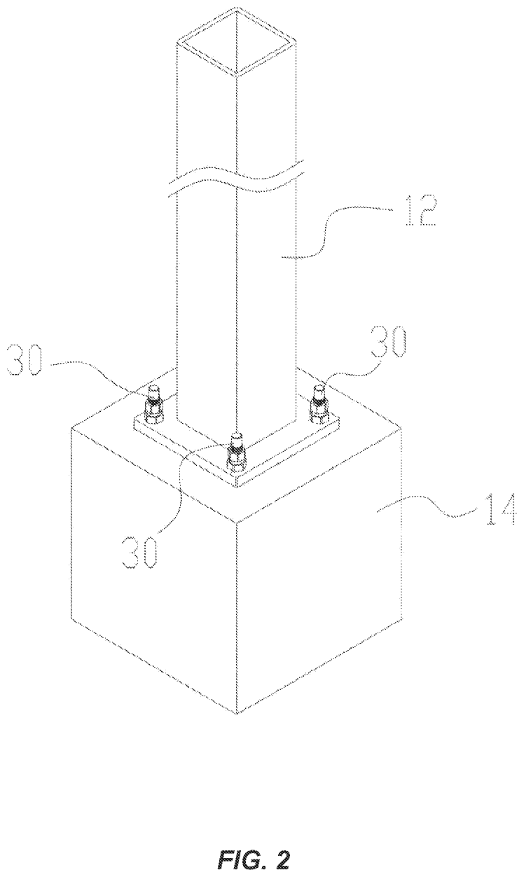

Specifically, as shown in FIG. 2, the anchor member 30 is applied to a joint site (anchor plate and end plate) to erect a column 12 configured to function as a prismatic steel material extending in the vertical direction of the structure 10 on the foundation 14. The anchor member 30 is embedded in the foundation 14. Of course, the embedding direction is not limited to the vertical direction and may be a horizontal direction or an oblique direction.

In addition, the anchor member 30 may have a length reaching the ground that supports the foundation 14, and may be an anchor that penetrates the foundation 14 to be directly embedded in the ground below. That is, the anchor member 30 is a coupling member configured to couple the solidified body and the structural member, and is used to join (fix) a structural material (framework material) of the structure 10 to the side of the foundation 14. In this way, the anchor member 30 may indirectly receive internal stress generated in the structural material by involving in the joining between the foundation 14 and the structural material.

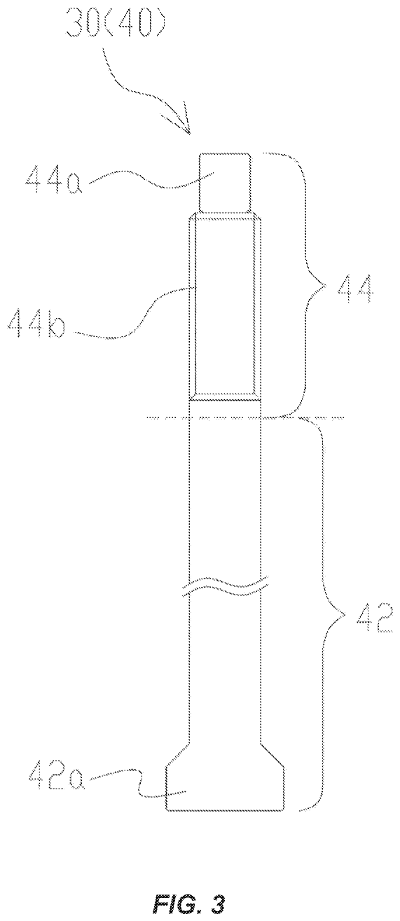

FIG. 3 illustrates a basic structure of an anchor bolt 40 as the anchor member 30. The anchor bolt 40 includes a fixing part having one end embedded in the foundation 14 and/or the ground and configured to fix the structural member to the other end side extending outside the foundation 14. Specifically, the anchor bolt 40 includes an embedded part 42 embedded in the foundation 14 and/or the ground, and a shaft part 44 (fixing part) configured to protrude more upward (to the outside of the foundation 14) than the foundation 14 to fix the other member by screwing. The embedded part 42 has a cylindrical shape, and has the head 42a whose end portion has an enlarged diameter.

In addition, in order to stand against the tensile strength of the anchor bolt 40, the embedded part 42 may be formed in the outer peripheral surface in a concave-convex shape. For example, the concave-convex shape may be formed in an appropriate shape, such as forming the concave-convex shape by a node extending in the circumferential direction of a deformed bar or by a screw node of a screw node reinforcing bar.

A cylindrical part 44a and a screw part 44b are formed in the shaft part 44, and the cylindrical part 44a is disposed on the tip end side. In addition, in the screw part 44b, the outer diameter or effective diameter of the male screw is set to be comparable to the outer diameter of the embedded part 42, but is not particularly limited.

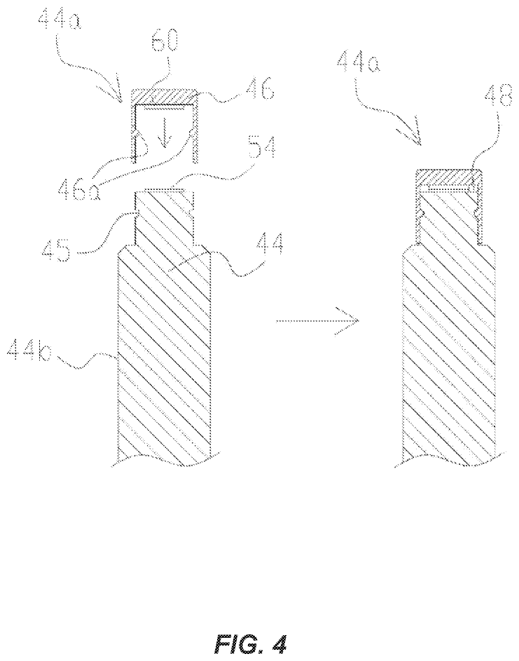

As shown in FIG. 4, the cylindrical part 44a is configured by mounting a cap 46 to an end of the shaft part 44. A mounting mechanism, configured to detachably mount the cap 46 to and from the shaft part 44, is formed between the end of the shaft part 44 and the cap 46. For example, the mounting mechanism includes a protrusion-shaped locking piece 46a formed on an inner peripheral surface of the cap 46 and a locking groove 45 formed on the outer peripheral surface of the end of the shaft part 44. Then, the cap 46 is mounted to the shaft part 44 by fitting the locking piece 46a to the locking groove 45. Of course, a screw-fitting structure may work as well.

In addition, an internal space 48 is formed in the cylindrical part 44a formed on an end surface of the shaft part 44 and inside the cap 46, and a terminal 54 or a circuit board 60 to be described later is disposed.

In addition, the anchor bolt 40 includes a conductive mechanism configured to detect stresses such as bending stress, compressive stress, and tensile stress applied to the bolt itself. Specifically, the anchor bolt 40 is configured by a sensor pattern and a conductive path directly disposed on the outer peripheral surface of the anchor bolt 40 and a terminal formed directly on the end surface of the shaft part 44.

Examples of formation of the sensor pattern, the conductive path, and the terminal will be described. For example, when a base material of the anchor bolt 40 has conductivity, an electrical insulation layer is formed on the surface of the anchor bolt 40, and a conductor configured to form patterns of the sensor pattern, the conductive path, and the terminal by a conductive material with favorable electrical conductivity on the electrical insulation layer.

The electrical insulation layer may be formed by the following examples including laminated print, pad print, painting, plating, inkjet print, sputtering, chemical vapor deposition (CVD), and physical vapor deposition (PVD). Alternatively, for example, the following methods may be applied, such as forming a film by sputtering an insulating material in a state in which a predetermined mask is placed, applying a silica material and then heat-treating the same, performing formation treatment, or forming a layer by an organic insulating material such as polyimide-based, epoxy-based, urethane-based, silicone-based, or fluorine-based materials.

When a base material of the anchor bolt 40 has electrical conductivity, a film, obtained via oxidation treatment of the surface of the base material, may be used as the electrical insulation layer. In addition, if the base material is aluminum-based, the electrical insulation layer may be installed by an alumite treatment. Of course, the electrical insulation layer is not limited to being formed by such methods. Moreover, when the base material of the anchor bolt 40 has electrical insulation, it is also possible to form the conductor configured to form the patterns of the sensor pattern, the conductive path, and the terminal directly on the base material without forming the electrical insulation layer.

The conductor is directly formed on the electrical insulation layer or the electrical insulation base material via lamination print, pad print, painting, plating, inkjet print, sputtering, CVD, and PVD using a conductive paste. In addition, the conductor may set the shape of a wiring by performing masking fit to the shape of the sensor pattern, the conductive path, and the terminal and then etching the same. In this way, by forming the conductor directly on the electrical insulation layer, the conductor is hardly peeled off for a long period of time. Of course, the sensor pattern, the conductive path, and the terminal may be formed in series on the anchor bolt 40.

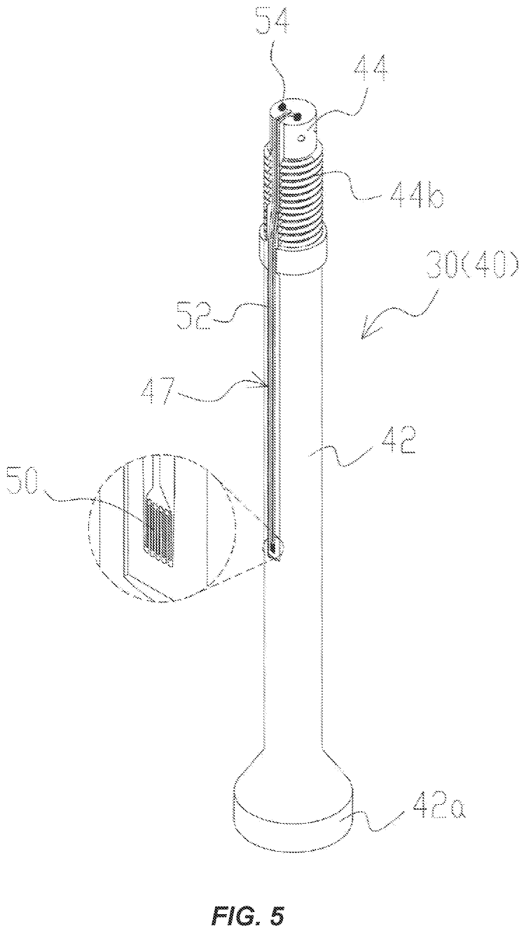

Next, with reference to FIG. 5, an example of the anchor bolt 40, which is configured to dispose the sensor pattern 50, the conductive path 52, and the terminal 54, will be described. In addition, FIG. 5 illustrates a state in which the cap 46 is separated to expose a cross section of the shaft part 44, as well as an enlarged portion where the sensor pattern 50 is disposed.

In FIG. 5, the sensor pattern 50 is arranged at the substantially central portion in the axial direction of the embedded part 42, and the conductive path 52 connected to the sensor pattern 50 is arranged by extending the same to the cross section of the shaft part 44. In addition, on the cross section of the shaft part 44, the terminal 54 is arranged to be connected to the conductive path 52.

The sensor pattern 50 includes a sensor structure portion extending by reciprocating a conductive material in the axial direction multiple times, and a lead portion extending from the sensor structure portion toward the shaft part 44. Accordingly, in the sensor pattern 50, electrical properties such as resistance value vary with the strain of the conductive material at the sensor structure portion. Detecting the change in the electrical properties, the sensor pattern 50 may be used as various sensors for detecting physical changes.

In addition, the physical changes detected by the change in the electrical properties may include heat/temperature change and humidity change. For example, when measuring an environmental temperature from the change in the electrical resistance value of the sensor pattern 50, it may refer to the use of the sensor pattern 50 as so-called structural components for resistance thermometers. Moreover, it is also possible to measure humidity by using the sensor pattern 50 as a resistance variable type electric humidity sensor. Such the sensor pattern 50 is conductively connected to the conductive path 52 formed on a side of the shaft part 44.

In addition, formed on the outer peripheral surfaces of the embedded part 42 and the shaft part 44 is a conductive path disposing part 47 in a concave shape whose cross section is non-circular. Regarding the conductive path disposing part 47, the bottom part of the concave cross section is planar, and the sensor pattern 50 and the conductive path 52 are directly formed in the bottom surface portion. Further, the extending direction of the conductive path disposing part 47 may be appropriately set such as extending in a direction inclined with respect to the axial direction on the outer peripheral surface if being in a series at least over the cross section of the shaft part 44. In addition, the depth and width of the conductive path disposing part 47 may be appropriately set as well.

By installing the conductive path disposing part 47, it is possible to form the conductor more easily than forming the conductor for the sensor pattern 50 and the conductive path 52 directly on the uneven surface of the anchor bolt 40.

As described above, by forming the sensor pattern 50, the conductive path 52, or the terminal 54 on the outer surface of the anchor bolt 40 as a pattern formation object, it is possible to obtain an elongated member having a sensing function without any issue even if the object is remarkably long.

Since the sensor pattern 50, the conductive path 52, and the terminal 54 described above are connected conductively, by connecting the terminal 54 to a circuit board (not shown), it is possible to acquire detection information based on the resistance value change in the sensor pattern 50 by means of an arithmetic circuit mounted on the circuit board. For example, IC chip or the like may be used as the circuit board.

The circuit board is installed to be in contact with the terminal 54 in the cylindrical part 44a, and an installation method may be appropriately set. For example, as shown in FIG. 4, while the circuit board 60 is mounted on the cap 46 in advance, the installation may be designed to be connected to the terminal 54 when the cap 46 is mounted to the shaft part 44.

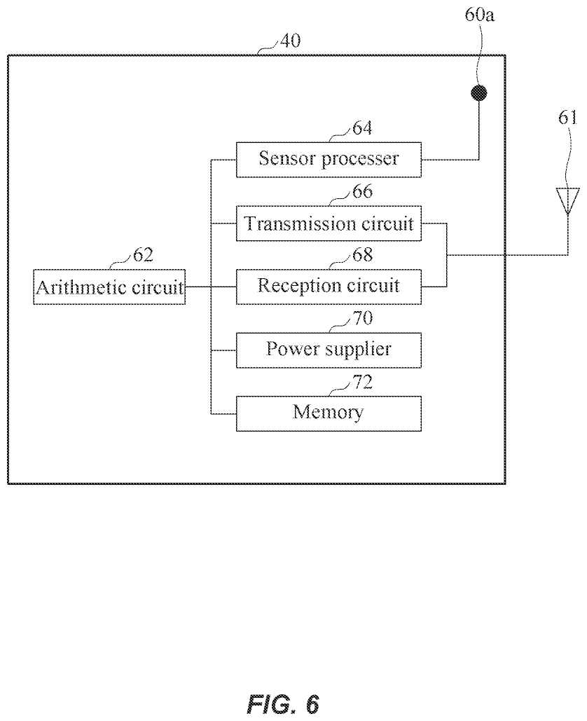

Here, with reference to the block diagram of FIG. 6, the configuration of the circuit board 60 mounted on the cap 46 will be described. The circuit board 60 mounted on the cap 46 includes a terminal 60a configured to be electrically connectable to the terminal 54 and an antenna 61 for wireless communication. Further, the circuit board 60 has an arithmetic circuit 62 that is connected with a sensor processer 64, a transmission circuit 66, a reception circuit 68, a power supplier 70, and a memory (72).

The sensor processer 64 includes a bridge circuit, an amplifier, and an A/D converter, and outputs detection information obtained by digitizing a detection signal derived by detecting a change in the resistance value of the sensor pattern 50. The transmission circuit 66 transmits the detection information transmitted from the sensor processer 64 to the outside via the antenna 61.

The reception circuit 68 receives various signals from the outside through the antenna 61. For example, the power supplier 70 is connected to an external power supply to supply power to each part of the circuit board 60. In the memory 72, the identifier ID assigned to each cap 46 and an initial resistance value of the sensor pattern 50 when no axial force is applied to the anchor bolt 40 are stored in advance, and detection information output from the sensor processer 64 is stored. Of course, the information stored in the memory 72 may be set appropriately, and is not particularly limited.

In addition, a method of supplying power to the power supplier 70 from the outside may include supplying power from a source in which a battery, a storage battery, or a photovoltaic power generation element is built, transmitting power through a wire such as an electric wire or the like, or wirelessly transmitting power through the antenna 61. As for the method by wireless power transmission, any method, such as “electromagnetic induction method,” “magnetic resonance method,” and “microwave method,” may be used and appropriately set according to the use environment.

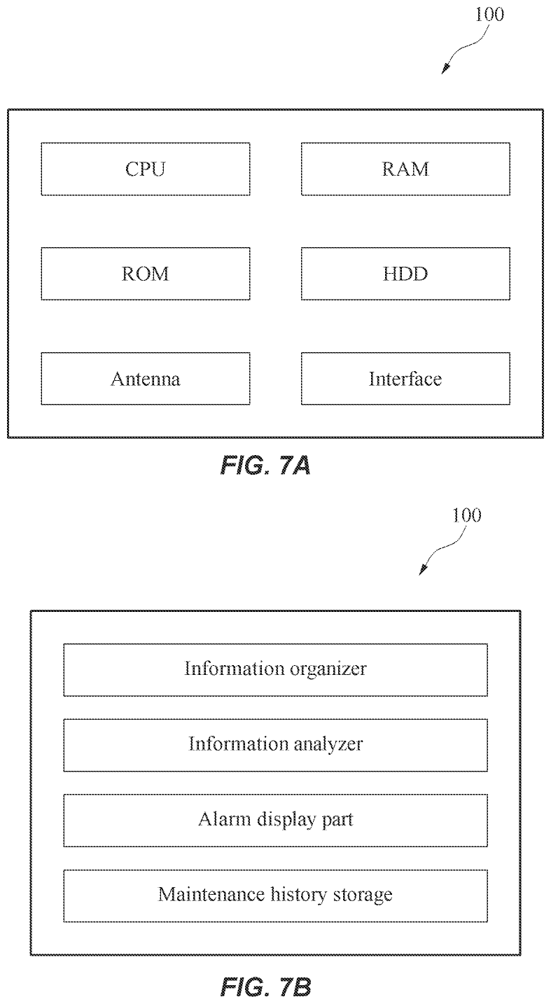

A hardware configuration of the information collection device 100 is shown in (A) in FIG. 7. The information collection device 100 is a server, including a CPU configured to function as a central processing device, a high-speed memory RAM configured to read and write temporary data, a read-only memory ROM configured to be used to store a mainboard program, a hard disk HDD configured to be writable to store data, an interface configured to perform external communication control, and an antenna configured to wirelessly communicate with the anchor bolt 40. In addition, the antenna is not limited to being arranged in the server configuring the hardware of the information collection device 100, and a relay antenna arranged in the vicinity of the anchor bolt 40 of each structure 10 may be appropriate.

A program configuration of the information collection device 100 is shown in (B) in FIG. 7. The information collection device 100 includes an information organizer, an information analyzer, an alarm display part, and a maintenance history storage. The information organizer is configured to accumulate, corresponding to the previously described individual identification information of the anchor bolt 40, various data such as resistance value data, acceleration data, temperature data, and displacement data collected from each anchor bolt 40 in time series, in addition to the name of the structure 10, an address, the installation site of the structural body, the installation direction, the size of the anchor member 30, and a manager (contact information).

The information analyzer is configured to analyze the collected various data to determine abnormality. For example, abnormality judgment is to analyze and determine whether abnormal numerical values appear with the passage of time or whether the mechanical balance of the entire structure 10 is not broken based on data collected from the plurality of anchor members 30. The alarm display part is configured to notify an operator of a maintenance alarm through a screen, text, or sound, when the information analyzer determines that abnormal data is included in the analysis result. The maintenance history storage is configured to store the maintenance history of the structure 10.

As described above, according to the measurement system 1 of the structure 10, the stress, strain, and/or displacement generated in the anchor member 30 may be detected by using the plurality of anchor members 30 for joining a structural body of the structure 10. The detection result is collected by the information collection device 100 through a wired or wireless connection so as to be utilized as objective data. In addition, for example, data collection may be automated, and observation and collection may be enabled in substantially real time, and the strain amount of the structure 10 and changes in internal stress may be detected when an earthquake or the like occurs. Based on the situation, it is also possible to determine the priority of maintenance and an important portions.

In addition, by forming the sensor pattern 50 directly in the embedded part 42, it is possible to grasp what kind of stress acts on the anchor bolt at the portion embedded in the concrete foundation or the ground, and also to determine resistance force and intensity of the foundation of the structure 10 or the ground.

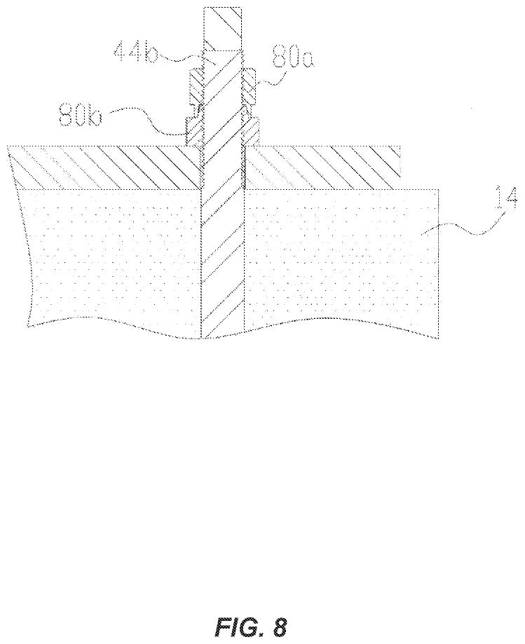

Further, although the method of fastening the anchor member 30 is various, it is preferable that the anchor bolt 40 has a structure that is never loosened as for the purpose of the measurement system 1. As an example of the structure, as shown in FIG. 8, the anchor bolt 40 that is never loosened may be prepared by forming two types of male screw helical grooves in the screw part 44b of the anchor bolt 40, screwing a first female screw body a first female screw body 80a screwed with one helical groove with a second female screw body 80b screwed with the other helical groove, and creating and placing a mechanism configured to prevent the relative rotation of both. Moreover, regarding the technique, please refer to Japan Patent No. 4663813, related to the present disclosure.

In addition, for example, using a ratchet mechanism or the like in which teeth are arranged on seating surfaces that face each other, the first female screw body 80a and the second female screw body 80b resist against the torque upon the application of torque in a release direction to the first female screw body 80a as the teeth are engaged with each other, thereby preventing relative rotation of the second female screw body 80b and the first female screw body 80a.

In addition, although the case that the sensor pattern 50 is formed directly in the embedded part 42 of the anchor bolt 40 is illustrated in the example embodiment, another structure may be adopted. For example, the sensor pattern 50 may be formed in the screw part 44b of the anchor bolt 40, and the sensor pattern 50 may be formed in each of the embedded part 42 and the screw part 44b. In that case, the conductive path 52 and the terminal 54 are installed in each sensor pattern 50. Of course, the number of sensor patterns 50 is not limited to one or two, but may be three or more. The sensor patterns 50 may be arranged at approximately equal intervals along a circumferential direction of the anchor bolt 40 when disposing the plurality of sensor patterns 50.

In addition, the disposing site of the sensor pattern 50 is not particularly limited. However, rather than the site embedded in the foundation 14, particularly a site protruding from the outside of the foundation 14 may make it easier to detect the stress applied to the anchor bolt 40. In addition, if the sensor pattern 50 is disposed around the boundary portion (referred to as a ‘boundary peripheral portion’) between the foundation 14 and the outside of the foundation 14, the load applied to the anchor bolt 40 embedded in the foundation 14 may be accurately grasped, compared to a case just disposing in the approximately central portion of the embedded part 42.

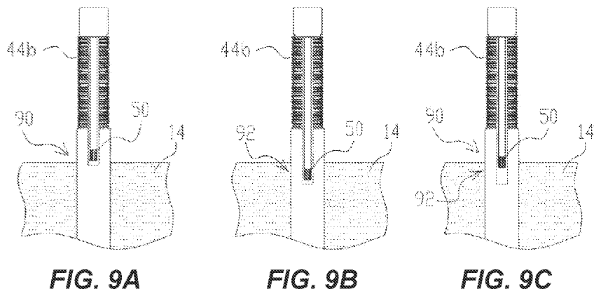

Accordingly, as shown in (A) of FIG. 9, the sensor pattern 50 may be positioned in the surface layer region 90 at the boundary peripheral portion. In addition, the boundary peripheral portion herein is not limited to the outside of the foundation 14, but includes the area inside the foundation 14. As shown in (B) in FIG. 9, the sensor pattern 50 may be positioned in an inner region 92 inside the foundation 14 of the boundary peripheral portion, and the sensor pattern 50 may be positioned to be engaged in two regions including the surface layer region 90 and the inner region 92 as shown in (C) of FIG. 9.

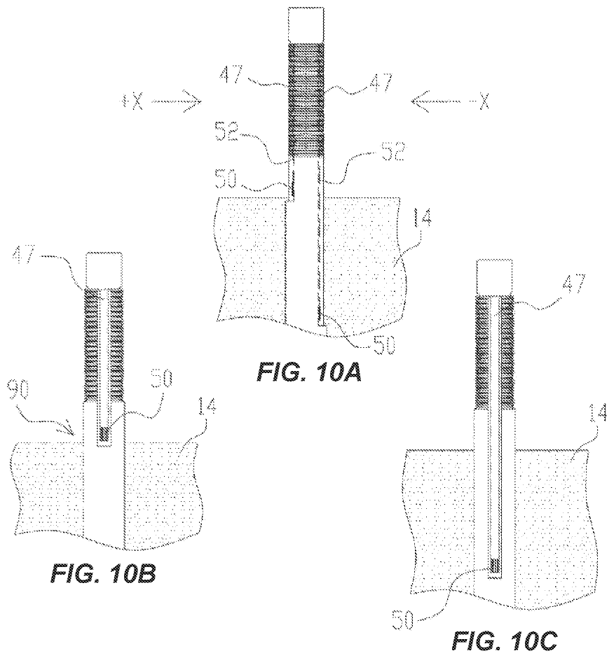

Of course, by preparing two sets of the sensor pattern 50, the conductive path 52 and the terminal 54, one set may be arranged in the boundary peripheral portion, and the other in the central portion (or near the head 42a) in the axial direction of the embedded part 42. Specifically, as shown in (A) of FIG. 10, installation of a pair of conductive path disposing parts 47 to face each other with an axial center of the anchor bolt 40 interposed therebetween may make the position of the sensor pattern 50 different in one conductive path disposing part 47 viewed from the arrow +X direction and in the other conductive path disposing part 47 viewed from the arrow −X direction. That is, in the conductive path disposing part 47 viewed from the arrow +X direction, as shown in (B) of FIG. 10, the sensor pattern 50 is arranged in the surface layer region 90. In the conductive path disposing part 47 viewed from the arrow −X direction, as shown in (C) of FIG. 10, the sensor pattern 50 is arranged in the vicinity of the center portion in the axial direction of the embedded part 42 in the foundation 14.

In this way, by changing the mutual axial positions of the two sensor patterns 50 so as to make one side placed on the outside of the foundation 14 and the other side on the inside of the foundation 14, it is possible to detect stress applied to the anchor bolt 40 and distortion as well as occurrence of an abnormality inside the foundation 14. Specifically, if there is no abnormality occurring in the foundation 14, only one sensor pattern 50 detects stress and distortion. Moreover, stress and distortion detected by the other sensor pattern 50 may be recognized due to expansion and contraction of the embedded part embedded in the foundation 14 by an external force. In addition, it is also possible to determine the occurrence of abnormalities such as separation between the foundation 14 and the anchor bolt 40 as well as peeling and destruction inside the foundation 14.

The example embodiments of the present disclosure are not limited to the above embodiments, and various modifications may be made within the scope not departing from the gist of the present disclosure.

1. A coupling member for coupling a solidified body and a structural member, the coupling member comprising:
a sensor part configured to be capable of measuring a physical change due to an external force and detect information that helps determine abnormalities in the solidified body and/or the structural member,
wherein the coupling member comprises an embedded part at one end that is embedded in the solidified body and/or a ground, and a fixing part configured to be capable of fixing the structural member to the other end side extending outside the solidified body.

2. The coupling member of claim 1, wherein the sensor part is installed in a surface layer region of a boundary peripheral portion of the solidified body and the outside of the solidified body.

3. The coupling member of claim 2, wherein the sensor part is installed in a region inside the solidified body of a boundary peripheral portion of the solidified body and the outside of the solidified body.

4. A measurement system, comprising:
the coupling member of claim 3; and
an information collection device that is connected to the sensor part by wire or wirelessly and configured to accumulate measurement information measured by the sensor part and determine abnormality in the solidified body and/or the structural member based on the measurement information.

5. The coupling member of claim 1, wherein the sensor part is installed in a region inside the solidified body of a boundary peripheral portion of the solidified body and the outside of the solidified body.

6. A measurement system, comprising:
the coupling member of claim 1; and
an information collection device that is connected to the sensor part by wire or wirelessly and configured to accumulate measurement information measured by the sensor part and determine abnormality in the solidified body and/or the structural member based on the measurement information.
